(19) United States
(12) Patent Application Publication
(10) Pub. No.: US 2022/0064608 A1
(43) Pub. Date: Mar. 3, 2022

(54) RECOMBINANT BACILLUS SUBTILIS STRAIN FOR PRODUCING UDP-GLYCOSYLTRANSFERASE AND RECOMBINATION METHOD THEREFOR

(71) Applicant: JIANGSU SHIYUTIAN BIOTECHNOLOGY CO., LTD., Wuxi (CN)

(72) Inventor: Yujie Chen, Wuxi (CN)

(21) Appl. No.: 17/531,703

(22) Filed: Nov. 19, 2021

Related U.S. Application Data
(63) Continuation of application No. PCT/CN2019/095986, filed on Jul. 15, 2019.

(30) Foreign Application Priority Data
May 20, 2019 (CN) 201910416745.9

(51) Int. Cl. C12N 9/10; C12N 15/75

(52) U.S. Cl. CPC C12N 9/1051; C12N 15/75

(57) ABSTRACT

The present invention discloses a recombinant Bacillus subtilis strain for producing UDP-glycosyltransferase and a recombination method therefor. The recombination method includes the following steps: chemically synthesizing a UDP-glycosyltransferase gene UGT and linking the UGT with a vector pUC57 to obtain pUC57-UGT, and cloning various promoters; linking the obtained pUC57-UGT and each of the promoters to an expression vector to obtain recombinant plasmids; transforming the obtained recombinant plasmids to host strains, respectively, to obtain recombinant plasmids of the host strains; respectively transforming the obtained recombinant plasmids of the host strains to Bacillus subtilis to obtain recombinant strains; and screening out recombinant Bacillus subtilis strain highly expressing UDP-glycosyltransferase from the obtained recombinant strains.

### CROSS-REFERENCE TO RELATED APPLICATIONS

The present application is a continuation application of PCT application No. PCT/CN2019/095986 filed on Jul. 15, 2019, which claims the benefit of Chinese Patent Application No. 201910416745.9 filed on May 20, 2019. The contents of the above identified applications are incorporated herein by reference in their entirety.

### REFERENCE TO SEQUENCE LISTING

The Sequence Listing is submitted as an ASCII formatted text file via EFS-Web, with a file name of “Sequence_Listing.TXT”, a creation date of Nov. 18, 2021, and a size of 54,331 bytes. The Sequence Listing filed via EFS-Web is part of the specification and is incorporated in its entirety by reference herein.

### TECHNICAL FIELD

The present invention relates to the field of genetic engineering, and in particular to a recombinant Bacillus subtilis strain for producing UDP-glycosyltransferase, and a recombination method therefor.

### BACKGROUND ART

UDP-glycosyltransferase may catalyze a reaction of rebaudioside A with UDP-glucose to generate rebaudioside D. Rebaudiosides A and D are constituents generating sweetness in stevioside, a novel sweetening agent. The sweetness of stevioside is several times higher than that of sucrose; but stevioside is basically non-caloric, and thus is widely used in the manufacture of food with reduced calorie. Stevioside is mainly extracted from Stevia rebaudiana. However, the extraction process is complex and needs lots of organic reagents, as well as having low yield.

Bacillus subtilis is a species of bacillus, which is a gram-positive bacterium, and will not produce endotoxin and other heat allergic proteins specifically produced by gram negative bacteria. Bacillus subtilis has been used for the preparation of fermented food for a long time, is a nonpathogenic bacterium and has been approved a safe food-grade strain by Food and Drug Administration. With the development of synthetic biology, it becomes possible to produce stevioside by means of microbial synthesis or enzyme catalysis.

### SUMMARY

The technical problem to be mainly solved by the present invention is to provide a recombinant Bacillus subtilis strain for producing UDP-glycosyltransferase, and a recombination method therefor. A UDP-glycosyltransferase gene is linked with various promoters, and then transformed into a host strain, and then genetically engineered bacteria capable of efficiently expressing UDP-glycosyltransferase are screened out.

To solve the above technical problem, one technical solution adopted by the present invention is as follows: providing a recombinant Bacillus subtilis strain for producing UDP-glycosyltransferase; recombinant bacterium expressing the UDP-glycosyltransferase gene is obtained by ligating UDP-glycosyltransferase gene together with various promoters onto an expression vector, followed by transforming to a host strain of Bacillus subtilis to construct recombinant strain of the UDP-glycosyltransferase gene.

In a preferred embodiment of the present invention, the UDP-glycosyltransferase gene is from Lycium chinensis. UDP-glycosyltransferase gene is abbreviated for UGT.

In a preferred embodiment of the present invention, the host strain is one of Bacillus subtilis (B. subtilis) 168, WB600, WB700, WB800 or progeny cells of the above strains.

In a preferred embodiment of the present invention, the promoters are selected from P_{hpaII}, Pₚ₄₃ and Pₚ₄₃ₜ; the P_{hpaII} is cloned from a plasmid pMA5, and the Pₚ₄₃ and the Pₚ₄₃ₜ are cloned from a Bacillus subtilis genome.

To solve the above technical problem, another technical solution adopted in the present invention is to: provide a recombination method for preparing a recombinant Bacillus subtilis strain for producing the above UDP-glycosyltransferase, including the following steps:

1) chemically synthesizing a UDP-glycosyltransferase gene UGT, linking the UGT with a vector pUC57 to obtain pUC57-UGT, and providing various promoters by cloning;

2) linking the pUC57-UGT obtained in the step 1) and each of the various promoters onto an expression vector to obtain a recombinant plasmid;

3) respectively transforming each of the recombinant plasmids obtained in the step 2) to Bacillus subtilis to obtain a recombinant strain; and

4) screening out a recombinant Bacillus subtilis strain highly expressing UDP-glycosyltransferase from the recombinant strains obtained in the step 3).

In a preferred embodiment of the present invention, a recombination method for preparing the above recombinant Bacillus subtilis strain for producing the above UDP-glycosyltransferase, includes the following steps:

1) chemically synthesizing the UDP-glycosyltransferase gene UGT and linking the UGT with the vector pUC57 to obtain pUC57-UGT; cloning the promoter P_{hpaII} from a plasmid pMA5, and cloning the promoters Pₚ₄₃ and the Pₚ₄₃ₜ from a Bacillus subtilis genome;

2) linking the pUC57-UGT obtained in the step 1) to the expression vector pMA5 pMA5 alone or together with each of the promoters P_{hpaII}, Pₚ₄₃ and Pₚ₄₃ₜ respectively, to obtain corresponding one of recombinant plasmids pMA5-UGT, pMA5-HpaII-UGT, pMA5-P43-UGT and pMA5-P43t-UGT;

3) amplifying P_{hpaII}-ugt, 2P_{hpaII}-ugt, P_{hpaII}-ₚ₄₃-ugt or P_{hpaII}-ₚ₄₃ₜ-ugt from the recombinant plasmids obtained in the step 2) as a template, respectively; linking each the above obtained P_{hpaII}-ugt, 2P_{hpaII}-ugt, P_{hpaII}-ₚ₄₃-ugt or P_{hpaII}-ₚ₄₃ₜ-ugt with a vector pMutin on which upstream and downstream gene fragments of a to-be-integrated site are linked, so that recombinant plasmids pMutin-HpaII-UGT, pMutin-2HpaII-UGT, pMutin-HpaII-P43-UGT and pMutin-HpaII-P43t-UGT are obtained;

4) respectively transforming the recombinant plasmids pMutin-HpaII-UGT, pMutin-2HpaII-UGT, pMutin-HpaII-P43-UGT and pMutin-HpaII-P43t-UGT obtained in the step 3) to Bacillus subtilis strains to obtain recombinant strains; and

5) screening out a recombinant Bacillus subtilis strain highly expressing UDP-glycosyltransferase from the recombinant strains obtained in the step 4).

In a preferred embodiment of the present invention, the UDP-glycosyltransferase gene UGT in the step 1) is obtained by performing codon optimization of UDP-glycosyltransferase gene UGT from Lycium chinensis and then subjecting to chemical synthesis.

In a preferred embodiment of the present invention, in the step 3), by using each of the pMA5-based recombinant plasmids obtained in the step 2) as templates, and using a primer pair ma-MuF/R, amplification is performed to obtain P_{hpaII}-ugt, 2P_{hpaII}-ugt, P_{hpaII}-ₚ₄₃-ugt or P_{hpaII}-ₚ₄₃ₜ-ugt; then linking each of the above obtained P_{hpaII}-ugt, 2P_{hpaII}-ugt, P_{hpaII}-ₚ₄₃-ugt or P_{hpaII}-ₚ₄₃ₜ-Ugt with a linear plasmid pMutin which is amplified using a primer MutinF/R and contains upstream and downstream fragments of to-be-integrated site amyE, thus obtaining the corresponding one of recombinant plasmids pMutin-HpaII-UGT, pMutin-2HpaII-UGT, pMutin-HpaII-P43-UGT and pMutin-HpaII-P43t-UGT.

The present invention has the following beneficial effects: the present invention achieves the expression of UDP-glycosyltransferase in a Bacillus subtilis strain by using a promoter screened out from various promoters.

### BRIEF DESCRIPTION OF DRAWINGS

To describe the technical solutions in the examples of the present invention more clearly, the accompanying drawings required in the description of the examples will be introduced simply. Apparently, the accompanying drawings described hereafter are merely some examples of the present invention. A person skilled in the art can further obtain other drawings according to these accompanying drawings without any inventive effort, where:

FIG. 1 shows the expression and production of a UGT enzyme in supernatant of fermentation broth when a bacillus strain W5-H-UGT is fermented for equal to or longer than 24 h;

FIG. 2 shows a HPLC chromatogram of production of rebaudioside D (RD) by enacymic conversion of rebaudioside A (RA) with the enzyme in 100 ml concentrated supernatant of fermentation broth when bacillus strain W5-H-UGT is fermented for equal to or longer than 48 h.

### DETAILED DESCRIPTION OF EMBODIMENTS

The technical solutions in the examples of the present invention will be described clearly and completely hereafter. Apparently, the examples described here are merely a portion of examples, not all embodiments of the present invention. Based on the examples of the present invention, all the other examples obtained by a person skilled in the art without any inventive effort shall fall within the scope set forth in the present invention.

Experimental methods in the following examples, unless other specified, are conventional methods. Unless otherwise specified, the materials, reagents and the like used in the examples are available commercially.

In the following examples, Bacillus subtilis WB600 is a nonpathogenic bacterium, and has advantages of clear genetic background, short generation time, easy culture and cheap raw materials of medium. The above biomaterials are merely used to repeat relevant experiments of the present invention, and may not be used as other purposes.

In the present invention the expression of UDP-glycosyltransferase in Bacillus subtilis strain is optimized by attempting a combination of various promoters.

### Example 1: Cloning of a UDP-Glycosyltransferase Gene

A codon optimized UDP-glycosyltransferase gene UGT from Lycium chinensis is obtained by chemical synthesis. Afterwards, the UDP-glycosyltransferase gene UGT is linked with a vector pUC57-Simple to obtain pUC57-UGT (UDP-glycosyltransferase has a nucleotide sequence of SEQ ID No. 1; and UDP-glycosyltransferase has an amino acid sequence of SEQ ID No. 2).

### Example 2: Construction of an Expression Vector

2.1. Construction of an Expression Vector pMA5-UGT

The UGT gene is amplified by using pUC57-UGT obtained in Example 1 as a template, and a primer pair Ugtma5F/Ugtma5BR (Table 1). The obtained PCR fragment was mixed with an expression vector pMA5 which was treated via double enzyme digestion with NdeI and BamHI (NEB Company) according to a certain ratio, then added to a Gibson reagent for reaction at 50° C. for 1 h. The above obtained ligation product was transformed to E. coli DH5a competent cells, and coated on a LB solid plate with 100 μg/mL carbenicillin, and then PCR identification was performed to screen out positive clones, followed by sequencing. the expression vector pMA5-UGT has the sequence of SEQ ID No. 3.

2.2. Construction of an Expression Vector pMA5-HpaII-UGT

The UGT gene is amplified from pUC57-UGT obtained in Example 1 as a template with a primer pair H-UgtF/Ugtma5BR (Table 1); P_{hpaII} promoter fragment is amplified from a plasmid pMA5 as a template with a primer pair H-ma5F/Hpa-UgtR (Table 1) (the P_{hpaII} promoter fragment has a nucleotide sequence of SEQ ID No. 3). The above obtained PCR fragment was mixed with an expression vector pMA5 which was treated via double enzyme digestion with NdeI and BamHI (NEB Company) according to a ratio of 1:1-5:1, then added to a Gibson reagent for reaction at 50° C. for 1 h. The above obtained linking product was transformed to E. coli DH5a competent cells, and coated on a LB solid plate with 100 μg/mL carbenicillin, then PCR identification was performed to screen out positive clones, followed by sequencing. The expression vector pMA5-HpaII-UGT has the sequence of SEQ ID No. 4.

2.3. Construction of an Expression Vector pMA5-P43-UGT

The UGT gene was amplified from the pUC57-UGT obtained in Example 1 as a template with a primer pair P43-UgtF/Ugtma5BR (Table 1). A Pₚ₄₃ promoter fragment is amplified from a Bacillus subtilis genome as a template with a primer pair HP43-ma5F/HP43-UgtR (Table 1) (the Pₚ₄₃ promoter fragment has a nucleotide sequence of SEQ ID No. 4). The above obtained PCR fragment was mixed with an expression vector pMA5 which was treated via double enzyme digestion with NdeI and BamHI (NEB Company) according to a ratio of 1:1-5:1, then added to a Gibson reagent for reaction at 50° C. for 1 h. The above obtained linking product was transformed to E. coli DH5α competent cells, and coated on a LB solid plate with 100 μg/mL carbenicillin, then PCR identification was performed to screen out positive clones and sequencing was performed. The expression vector pMA5-P43-UGT has the sequence of SEQ ID No. 5:

2.4. Construction of an Expression Vector pMA5-P43t-UGT

The UGT gene was amplified from the pUC57-UGT obtained in Example 1 as a template with a primer pair P43t-UgtF/Ugtma5BR (Table 1). A truncated Pₚ₄₃ promoter fragment_Pₚ₄₃ₜ was amplified from a Bacillus subtilis genome as a template with a primer pair HP43-ma5F/HP43t-UgtR (Table 1) (the Pₚ₄₃ₜ promoter fragment has a nucleotide sequence of SEQ ID No. 5). The above obtained PCR fragment was mixed with an expression vector pMA5 which was treated via double enzyme digestion with NdeI and BamHI (NEB Company) according to a ratio of 1:1-5:1, then added to a Gibson reagent for reaction at 50° C. for 1 h. The above obtained linking product was transformed to E. coli DH5α competent cells, and coated on a LB solid plate with 100 μg/mL carbenicillin, then PCR identification was performed to screen out positive clones and sequencing was performed. The expression vector pMA5-P43t-UGT has the sequence of SEQ ID No. 6.

2.5. Construction of pMutin-Based Vectors

Each of the pMA5-based plasmids obtained in example 2.1-2.4 served as a template, and a primer pair ma-MuF/R (Table 1) was used to amplify P_{hpaII}-ugt, 2P_{hpaII}-ugt, P_{hpaII}-ₚ₄₃-ugt ₒᵣ P_{hpaII}-ₚ₄₃ₜ-ugt, respectively; then each of the above obtained P_{hpaII}-ugt, 2P_{hpaII}-ugt, P_{hpaII}-ₚ₄₃-ugt and P_{hpaII}-ₚ₄₃ₜ-ugt was linked with a linear plasmid pMutin which was amplified using a primer MutinF/R (Table 1) and contained upstream and downstream fragments of to-be-integrated site amyE, thus obtaining recombinant plasmids pMutin-HpaII-UGT, pMutin-2HpaII-UGT, pMutin-HpaII-P43-UGT and pMutin-HpaII-P43t-UGT.

TABLE 1
Sequence table of the primers

| Primer name | Sequence | |
|---|---|---|
| Ugtma5F | GGAGCGATTTACATATGGCAACAAATTTAAGAGTCCTG | (SEQ ID No. 7) |
| Ugtma5BR | GCTCGACTCTAGAGGATCCTTATTTTGATTTGTTAGAATTG | (SEQ ID No. 8) |
| H-UgtF | GGAGCGATTTACATATGGCAACAAATTTAAGAGTC | (SEQ ID No. 9) |
| H-ma5F | CTAAAAAGGAGCGATTTACATGATCTTCTCAAAAAATACTACC | (SEQ ID No. 10) |
| [symbol] -UgtR | GTTGCCATATGTAAATCGCTCCTTTTTAGGTG | (SEQ ID No. 11) |
| P43-UgtF | GGAATGTACACCATATGGCAACAAATTTAAGAGTC | (SEQ ID No. 12) |
| HP43- [symbol] | CTAAAAAGGAGCGATTTACATTGATAGGTGGTATGTTTTC | (SEQ ID No. 13) |
| HP- [symbol] -UtgR | GTTGCCATATGGTGTACATTCCTCTCTTACC | (SEQ ID No. 14) |
| P43T-UgtF | GATAGCGGTACCACATATGGCAACAAATTTAAGAGTC | (SEQ ID No. 15) |
| HP43t-UgtR | GTTGCCATATGTGGTACCGCTATCACTTTATA | (SEQ ID No. 16) |
| ma-MuF | CAAATAAGGAGTGTCAAGAGGCAAGGGTTTAAAGGTGGAG | (SEQ ID No. 17) |
| ma-MuR | GTCCCGTCTAGCCTTGCCCGAGCTCGACTCTAGAGGATCC | (SEQ ID No. 18) |
| MutinF | GGGCAAGGCTAGACGGGAC | (SEQ ID No. 19) |
| MutinR | TCTTGACACTCCTTATTTG | (SEQ ID No. 20) |

[symbol]

indicates data missing or illegible when filed

### Example 3: Construction of Recombinant Strains

Plasmids constructed in Example 2 were respectively transformed to Bacillus subtilis WB600 competent cells, correct recombinant strains are screened out, and then the recombinant strains were subjected to fermentation.

3.1. Preparation of Bacillus subtilis WB600 Competent Cells

Bacillus subtilis WB600 single colonies were picked from a fresh cultured LB plate (per liter contained 10 g tryptone, 5 g yeast powder and 10 g NaCl), inoculated onto a 3 mL LB medium and cultured overnight at 37° C. and 200 rpm. The overnight cultured product was inoculated onto a 100 mL NCM medium (per liter, containing 17.4 g K₂HPO₄, 11.6 g NaCl, 5 g glucose, 5 g peptone, 1 g yeast extract, 0.3 g sodium citrate dihydrate, 0.05 g MgSO₄.7H₂O, and 91.1 g sorbitol, pH=7.2) according to a ratio of 1:100, then cultured at 37° C. and 200 rpm to OD600=0.5. 3.89% glycine and 1.06% DL-threonine were added for continuous culture for 1 h. All the culture solution was treated in ice-water bath for 20 min, and were then centrifuged at 8000 g at 4° C. for 5 min to collect bacterial cells. The bacterial cells were washed with 10 mL pre-cooled electro-transfection buffer solution ETM (per liter, containing 0.5M sorbitol, 0.5M mannitol, 0.5M trehalose, 10% (v/v) glycerin, 0.25 mM K₂HPO₄, 0.25 mM KH₂PO₄, and 0.25 mM MgCl₂); then were centrifuged at 8000 g at 4° C. for 5 min to remove supernatant, and then repeating washing as described above 3 times. The washed bacterial cells were resuspended in 1 mL ETM, and subpackaged into 100 μL per tube, and freeze-preserved at −80° C. for further use.

3.2. Construction of Bacillus subtilis Recombinant Strain

1 μg of each of plasmids pMutin, pMutin-HpaII-UGT, pMutin-2HpaII-UGT, pMutin-HpaII-P43-UGT and pMutin-HpaII-P43t-UGT were added to 100 μL Bacillus subtilis WB600 competent cells, respectively, and incubated for 5 min in ice bath, then transferred to a pre-cooled 1 mm electric-revolving cup (Bio-Rad); and an electric-revolving instrument (Bio-Rad Micropulser) was used for electroporation for once at a voltage of 2.1 kv. At the end of electroporation, 1 mL resuscitation medium (NCM added with 0.38 M mannitol and 0.5 M trehalose) was immediately added, and electroporation was performed for 6 min at 46° C. and resuscitation was performed at 37° C. and 200 rpm for 3 h. Each of the bacteria solutions was poured into a LB solid medium plate added with 10 μg/mL erythromycin, respectively, put on a super clean bench and dried. Then the plate was put to 37° C. incubator for overnight culture. Positive clones were screened out by PCR, and single colonies were taken and incubated into an antibiotics-free LB medium. Strains without erythromycin resistance were screened out after through multiple subcultures. Positive clones were screened out by PCR again, thus obtaining recombinant Bacillus subtilis (B. subtilis) strains W5/W5-UGT/W5-H-UGT/W5-P-UGT/W5-Pt-UGT.

3.3. Culture of Recombinant Bacillus subtilis Strain

The activated recombinant Bacillus subtilis strain was incubated on a 1.5 L initial medium containing 1% casein peptone, 0.5% yeast powder, and 0.5% glycerin according to a ratio of 1:20; the temperature was controlled to 37° C., dissolved oxygen was maintained at 20% by rotational speed control; during fermentation process, glucose having a concentration of 40% was supplemented, and residual sugar was controlled not higher than 1 g/L, and materials were continuously supplemented in a fed-batch way, then pH was controlled to 7.4 with ammonium hydroxide, for culturing for 48 h. A defoamer was added according to the requirements.

### Example 4: Detection and Analysis on the Expression of UGT Protein and Enzyme Activity

Supernatant was collected from the fermentation broth of the recombinant Bacillus subtilis strain cultured in Example 3, and filtered to remove bacteria; and the protein was concentrated by ultrafiltration and, then SDS-PAGE was utilized to detect UGT expression, and the crude enzyme was subjected to enzyme activity detection analysis.

4.1. Detection on the UGT Protein Expression

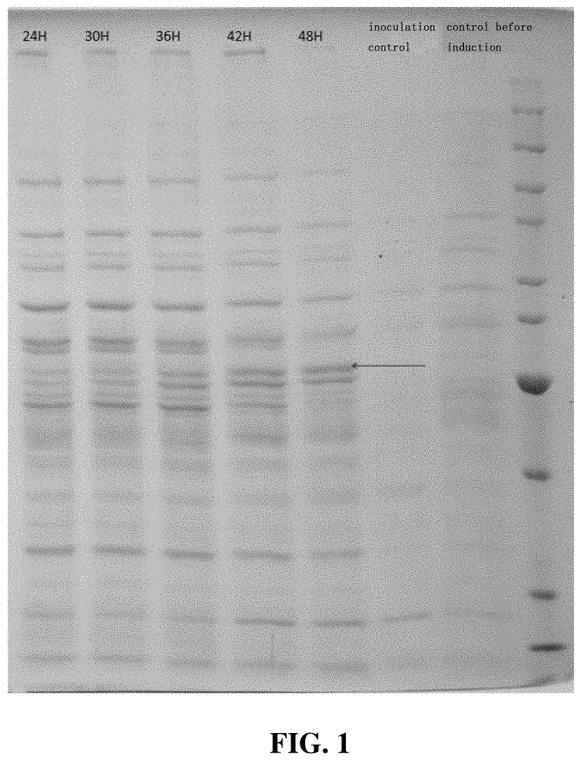

A centrifugal machine was used for centrifugation at 4° C. and 8000 rpm for 10 min to collect the fermentation broth of the recombinant Bacillus subtilis strain obtained in 3.3, and SDS-PAGE electrophoretic analysis was performed to analyze the expression of the UGT protein (Table 2). FIG. 1 shows that an enzyme produced in fermentation broth supernatant is detected by an SDS-PAGE gel, which indicates the expression and production of a UGT enzyme in fermentation broth supernatant when bacillus strain W5-H-UGT is fermented for equal to or longer than 24 h; where the arrow points protein bands of the recombinantly expressed Lycium chinensis UGT.

TABLE 2
Comparison of UGT expressed by various recombinant strains

| Strain | Protein expression |
|---|---|
| B. subtilis W5 | − |
| B. subtilis W5-UGT | ++ |
| B. subtilis W5-H-UGT | +++ |
| B. subtilis W5-P-UGT | + |
| B. subtilis W5-Pt-UGT | − |

Note:
“−” denotes no expression of protein, and
“+” denotes expression of protein.

4.2. Detection on the UGT Enzyme Activity

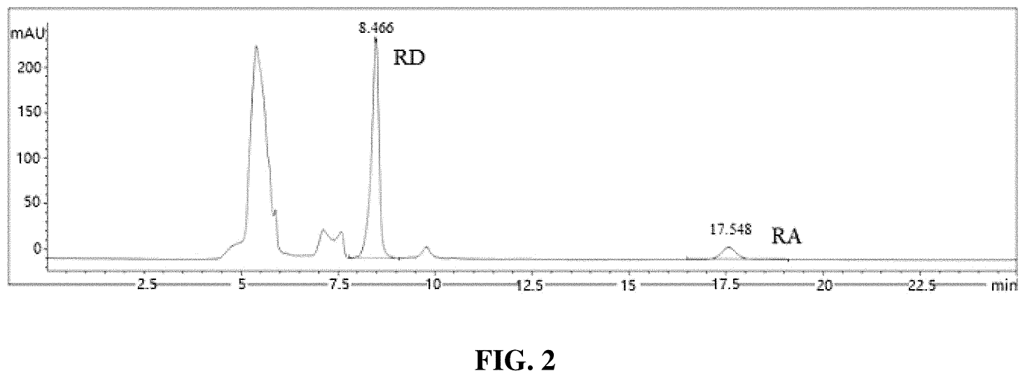

10 μL of the above crude enzyme in supernatant obtained from 4.1, 10 μL 20% rebaudioside A and 2 μL 1% UDP-glucose, were taken and added with double distilled water to 200 μL. The above solutions were mixed well and reacted at 37° C. for 1 h, then the reaction solution was treated in a boiling water bath for 5 min, and then centrifuged for 5 min with a centrifuge at 12000 rpm, and supernatant was taken to detect the production of rebaudioside D by HPLC. High-performance liquid chromatography was performed according to “A3.1 Method I” in “Determination of A3 Stevioside Determination” in GB8270-2014. FIG. 2 shows a HPLC chromatogram of production of rebaudioside D (RD) by enacymic conversion of rebaudioside A (RA) with the enzyme in 100 ml concentrated supernatant of fermentation broth when bacillus strain W5-H-UGT is fermented for equal to or longer than 48 h. As shown in FIG. 2, under the experimental conditions, 40 g/L of rebaudioside A can be converted into about 30 g/L of rebaudioside D, 8 h later.

In the present invention, recombinant strains are obtained by linking UDP-glycosyltransferase gene with various promoters and then transforming into Bacillus subtilis, and recombinant Bacillus subtilis strain capable of efficiently secreting and expressing UDP-glycosyltransferase was screened out. Moreover, the extracellularly secreted UGT can convert rebaudioside A to rebaudioside D.

What is mentioned above are merely examples of the present invention, but not thus construed as limiting the protection scope of the present invention. Any equivalent structure or flow transformation made by means of the description of the present invention, or direct or indirect application thereof in other related technical fields shall be similarly included in the protection scope of the present invention.

What is claimed is:

1. A recombinant Bacillus subtilis strain for producing UDP-glycosyltransferase, wherein the recombinant strain is obtained by expressing a UDP-glycosyltransferase gene in a microorganism, wherein the UDP-glycosyltransferase gene is linked to an expression vector together with each of various promoters, and then transformed to a host strain of Bacillus subtilis to construct the recombinant strain of the UDP-glycosyltransferase gene.

2. The recombinant Bacillus subtilis strain for producing UDP-glycosyltransferase according to claim 1, wherein the UDP-glycosyltransferase gene is from Lycium chinensis.

3. The recombinant Bacillus subtilis strain for producing UDP-glycosyltransferase according to claim 1, wherein the host strain is one of Bacillus subtilis (B. subtilis) 168, WB600, WB700, WB800 or progeny cells of the above strains.

4. The recombinant Bacillus subtilis strain for producing UDP-glycosyltransferase according to claim 1, wherein the promoters are selected from P_{hpaII}, Pₚ₄₃ and Pₚ₄₃ₜ; the P_{hpaII} is cloned from a plasmid pMA5, and the Pₚ₄₃ and the Pₚ₄₃ₜ are cloned from a Bacillus subtilis genome.

5. A recombination method for preparing a recombinant Bacillus subtilis strain for producing UDP-glycosyltransferase, comprising the following steps:
1) chemically synthesizing a UDP-glycosyltransferase gene UGT and linking the UGT with a vector pUC57 to obtain pUC57-UGT, and cloning various promoters;
2) linking the pUC57-UGT obtained in the step 1) and each of the promoters to an expression vector, respectively, to obtain corresponding one of recombinant plasmids;
3) transforming each of the recombinant plasmids obtained in the step 2) to a Bacillus subtilis strain, respectively, to obtain corresponding one of recombinant strains; and
4) screening out a recombinant Bacillus subtilis strain highly expressing UDP-glycosyltransferase from the recombinant strains obtained in the step 3).

6. The recombination method according to claim 5, characterized by comprising the following steps:
1) chemically synthesizing the UDP-glycosyltransferase gene UGT and linking the UGT with the vector pUC57 to obtain pUC57-UGT; cloning the promoter P_{hpaII} from a plasmid pMA5, and cloning the promoters Pₚ₄₃ and the Pₚ₄₃ₜ from a Bacillus subtilis genome;
2) linking the pUC57-UGT obtained in the step 1) to the expression vector pMA5 pMA5 alone or together with each of the promoters P_{hpaII}, Pₚ₄₃ and Pₚ₄₃ₜ respectively, to obtain corresponding one of recombinant plasmids pMA5-UGT, pMA5-HpaII-UGT, pMA5-P43-UGT and pMA5-P43t-UGT;
3) amplifying P_{hpaII}-ugt, 2P_{hpaII}-ugt, P_{hpaII}-ₚ₄₃-ugt or P_{hpaII}-ₚ₄₃ₜ-ugt from the recombinant plasmids obtained in the step 2) as a template, respectively; linking each the above obtained P_{hpaII}-ugt, 2P_{hpaII}-ugt, P_{hpaII}-ₚ₄₃-ugt or P_{hpaII}-ₚ₄₃ₜ-ugt with a vector pMutin on which upstream and downstream gene fragments of a to-be-integrated site are linked, so that recombinant plasmids pMutin-HpaII-UGT, pMutin-2HpaII-UGT, pMutin-HpaII-P43-UGT and pMutin-HpaII-P43t-UGT are obtained;
4) respectively transforming the recombinant plasmids pMutin-HpaII-UGT, pMutin-2HpaII-UGT, pMutin-HpaII-P43-UGT and pMutin-HpaII-P43t-UGT obtained in the step 3) to Bacillus subtilis strains to obtain recombinant strains; and
5) screening out a recombinant Bacillus subtilis strain highly expressing UDP-glycosyltransferase from the recombinant strains obtained in the step 4).

7. The recombination method according to claim 5, wherein the UDP-glycosyltransferase gene UGT in the step 1) is obtained by performing codon optimization of a UDP-glycosyltransferase gene UGT from Lycium chinensis followed by performing chemical synthesis.

8. The recombination method according to claim 5, wherein in the step 3), amplifying P_{hpaII}-ugt, 2P_{hpaII}-ugt, P_{hpaII}-ₚ₄₃-ugt or P_{hpaII}-ₚ₄₃ₜ-ugt from the pMA5-based recombinant plasmids obtained in the step 2) as a template, respectively, by using a primer pair ma-MuF/R; then linking each of the above obtained P_{hpaII}-ugt, 2P_{hpaII}-ugt, P_{hpaII}-ₚ₄₃-ugt or P_{hpaII}-ₚ₄₃ₜ-ugt with a linear plasmid pMutin which is amplified using a primer MutinF/R and contains upstream and downstream fragments of a to-be-integrated site amyE, to obtain recombinant plasmids pMutin-HpaII-UGT, pMutin-2HpaII-UGT, pMutin-HpaII-P43-UGT and pMutin-HpaII-P43t-UGT.


SEQUENCE LISTING (20 sequences)

SEQ ID NO: 1 - DNA, 1365 bp, Artificial Sequence
  Codon optimized UGT
atgggcaccgaggtgaccgttcacaaaaacaccctgcgtgtgctgatgttcccgtggctggcgtacggtcacatcagcccgtttctgaacgttgcgaagaaactggtggatcgtggcttcctgatctacctgtgcagcaccgcgattaacctgaaaagcaccatcaagaaaattccggagaagtatagcgacagcatccagctgattgaactgcacctgccggagctgccggaactgccgccgcactaccacaccaccaacggtctgccgccgcacctgaaccacaccctgcaaaaggcgctgaaaatgagcaagccgaacttcagcaaaatcctgcaaaacctgaagccggacctggttatttatgacctgctgcaacaatgggcggagggcgttgcgaacgaacaaaacattccggcggtgaagctgctgaccagcggtgcggcggttctgagctacttctttaacctggttaagaaaccgggcgtggagttcccgtttccggcgatttatctgcgtaaaaacgagctggaaaagatgagcgagctgctggcgcagagcgcgaaggataaagaaccggatggtgtggatccgtttgcggatggtaacatgcaagttatgctgatgagcaccagccgtatcatcgaagcgaagtacatcgattactttagcggtctgagcaactggaaggtggttccggtgggtccgccggttcaggacccgatcgcggacgatgcggacgagatggaactgattgattggctgggtaagaaagacgagaacagcaccgtgttcgttagctttggcagcgagtacttcctgagcaaagaagatcgtgaggaaatcgcgtttggtctggaactgagcaacgttaacttcatttgggtggcgcgttttccgaaaggcgaggaacagaacctggaggacgcgctgccgaagggcttcctggaacgtatcggtgatcgtggccgtgtgctggacaagtttgcgccgcaaccgcgtatcctgaaccacccgagcaccggtggcttcattagccactgcggttggaacagcgtgatggaaagcgttgattttggcgtgccgatcattgcgatgccgatccacctggaccagccgatgaacgcgcgtctgattgtggagctgggtgtggcggttgaaatcgttcgtgacgattatggcaaaattcaccgtgaggaaatcgcggagattctgaaagatgttatcgcgggcaagagcggcgaaaacctgaaggcgaaaatgcgtgacatcagcaaaaacctgaagagcattcgtgacgaggaaatggataccgcggcggaggaactgattcaactgtgcaagaacagcccgaagctgaaataa

SEQ ID NO: 2 - protein, 454 aa, Lycium chinensis
MGTEVTVHKNTLRVLMFPWLAYGHISPFLNVAKKLVDRGFLIYLCSTAINLKSTIKKIPEKYSDSIQLIELHLPELPELPPHYHTTNGLPPHLNHTLQKALKMSKPNFSKILQNLKPDLVIYDLLQQWAEGVANEQNIPAVKLLTSGAAVLSYFFNLVKKPGVEFPFPAIYLRKNELEKMSELLAQSAKDKEPDGVDPFADGNMQVMLMSTSRIIEAKYIDYFSGLSNWKVVPVGPPVQDPIADDADEMELIDWLGKKDENSTVFVSFGSEYFLSKEDREEIAFGLELSNVNFIWVARFPKGEEQNLEDALPKGFLERIGDRGRVLDKFAPQPRILNHPSTGGFISHCGWNSVMESVDFGVPIIAMPIHLDQPMNARLIVELGVAVEIVRDDYGKIHREEIAEILKDVIAGKSGENLKAKMRDISKNLKSIRDEEMDTAAEELIQLCKNSPKLK

SEQ ID NO: 3 - DNA, 8429 bp, Artificial Sequence
  pMA5-UGT
catatgggcaccgaggtgaccgttcacaaaaacaccctgcgtgtgctgatgttcccgtggctggcgtacggtcacatcagcccgtttctgaacgttgcgaagaaactggtggatcgtggcttcctgatctacctgtgcagcaccgcgattaacctgaaaagcaccatcaagaaaattccggagaagtatagcgacagcatccagctgattgaactgcacctgccggagctgccggaactgccgccgcactaccacaccaccaacggtctgccgccgcacctgaaccacaccctgcaaaaggcgctgaaaatgagcaagccgaacttcagcaaaatcctgcaaaacctgaagccggacctggttatttatgacctgctgcaacaatgggcggagggcgttgcgaacgaacaaaacattccggcggtgaagctgctgaccagcggtgcggcggttctgagctacttctttaacctggttaagaaaccgggcgtggagttcccgtttccggcgatttatctgcgtaaaaacgagctggaaaagatgagcgagctgctggcgcagagcgcgaaggataaagaaccggatggtgtggatccgtttgcggatggtaacatgcaagttatgctgatgagcaccagccgtatcatcgaagcgaagtacatcgattactttagcggtctgagcaactggaaggtggttccggtgggtccgccggttcaggacccgatcgcggacgatgcggacgagatggaactgattgattggctgggtaagaaagacgagaacagcaccgtgttcgttagctttggcagcgagtacttcctgagcaaagaagatcgtgaggaaatcgcgtttggtctggaactgagcaacgttaacttcatttgggtggcgcgttttccgaaaggcgaggaacagaacctggaggacgcgctgccgaagggcttcctggaacgtatcggtgatcgtggccgtgtgctggacaagtttgcgccgcaaccgcgtatcctgaaccacccgagcaccggtggcttcattagccactgcggttggaacagcgtgatggaaagcgttgattttggcgtgccgatcattgcgatgccgatccacctggaccagccgatgaacgcgcgtctgattgtggagctgggtgtggcggttgaaatcgttcgtgacgattatggcaaaattcaccgtgaggaaatcgcggagattctgaaagatgttatcgcgggcaagagcggcgaaaacctgaaggcgaaaatgcgtgacatcagcaaaaacctgaagagcattcgtgacgaggaaatggataccgcggcggaggaactgattcaactgtgcaagaacagcccgaagctgaaataaggatcctctagagtcgagctcaagctagcttggtacgtaccagatctgagatcacgcgttctagaggtcgaaattcacctcgaaagcaagctgataaaccgatacaattaaaggctccttttggagcctttttttttggagattttcaacgtgaaaaaattattattcgcaattccaagctaattcacctcgaaagcaagctgataaaccgatacaattaaaggctccttttggagcctttttttttggagattttcaacgtgaaaaaattattattcgcaattccaagctctgcctcgcgcgtttcggtgatgacggtgaaaacctctgacacatgcagctcccggagacggtcacagcttgtctgtaagcggatgcagatcacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgactgcaatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaagtactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggcatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacgaaattccggatgagcattcatcaggcgggcaagaatgtgaataaaggccggataaaacttgtgcttatttttctttacggtctttaaaaaggccgtaatatccagctaaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcgaagacgaaagggcatcgcgcgcggggaattcccgggagagctcgatatcgcatgcggtacctctagaagaagcttggagacaaggtaaaggataaaacagcacaattccaagaaaaacacgatttagaacctaaaaagaacgaatttgaactaactcataaccgagaggtaaaaaaagaacgaagtcgagatcagggaatgagtttataaaataaaaaaagcacctgaaaaggtgtctttttttgatggttttgaacttgttctttcttatcttgatacatatagaaataacgtcatttttattttagttgctgaaaggtgcgttgaagtgttggtatgtatgtgttttaaagtattgaaaacccttaaaattggttgcacagaaaaaccccatctgttaaagttataagtgactaaacaaataactaaatagatgggggtttcttttaatattatgtgtcctaatagtagcatttattcagatgaaaaatcaagggttttagtggacaagacaaaaagtggaaaagtgagaccatggagagaaaagaaaatcgctaatgttgattactttgaacttctgcatattcttgaatttaaaaaggctgaaagagtaaaagattgtgctgaaatattagagtataaacaaaatcgtgaaacaggcgaaagaaagttgtatcgagtgtggttttgtaaatccaggctttgtccaatgtgcaactggaggagagcaatgaaacatggcattcagtcacaaaaggttgttgctgaagttattaaacaaaagccaacagttcgttggttgtttctcacattaacagttaaaaatgtttatgatggcgaagaattaaataagagtttgtcagatatggctcaaggatttcgccgaatgatgcaatataaaaaaattaataaaaatcttgttggttttatgcgtgcaacggaagtgacaataaataataaagataattcttataatcagcacatgcatgtattggtatgtgtggaaccaacttattttaagaatacagaaaactacgtgaatcaaaaacaatggattcaattttggaaaaaggcaatgaaattagactatgatccaaatgtaaaagttcaaatgattcgaccgaaaaataaatataaatcggatatacaatcggcaattgacgaaactgcaaaatatcctgtaaaggatacggattttatgaccgatgatgaagaaaagaatttgaaacgtttgtctgatttggaggaaggtttacaccgtaaaaggttaatctcctatggtggtttgttaaaagaaatacataaaaaattaaaccttgatgacacagaagaaggcgatttgattcatacagatgatgacgaaaaagccgatgaagatggattttctattattgcaatgtggaattgggaacggaaaaattattttattaaagagtagttcaacaaacgggccagtttgttgaagattagatgctataattgttattaaaaggattgaaggatgcttaggaagacgagttattaatagctgaataagaacggtgctctccaaatattcttatttagaaaagcaaatctaaaattatctgaaaagggaatgagaatagtgaatggaccaataataatgactagagaagaaagaatgaagattgttcatgaaattaaggaacgaatattggataaatatggggatgatgttaaggctattggtgtttatggctctcttggtcgtcagactgatgggccctattcggatattgagatgatgtgtgtcatgtcaacagaggaagcagagttcagccatgaatggacaaccggtgagtggaaggtggaagtgaattttgatagcgaagagattctactagattatgcatctcaggtggaatcagattggccgcttacacatggtcaatttttctctattttgccgatttatgattcaggtggatacttagagaaagtgtatcaaactgctaaatcggtagaagcccaaacgttccacgatgcgatttgtgcccttatcgtagaagagctgtttgaatatgcaggcaaatggcgtaatattcgtgtgcaaggaccgacaacatttctaccatccttgactgtacaggtagcaatggcaggtgccatgttgattggtctgcatcatcgcatctgttatacgacgagcgcttcggtcttaactgaagcagttaagcaatcagatcttccttcaggttatgaccatctgtgccagttcgtaatgtctggtcaactttccgactctgagaaacttctggaatcgctagagaatttctggaatgggattcaggagtggacagaacgacacggatatatagtggatgtgtcaaaacgcataccattttgaacgatgacctctaataattgttaatcatgttggttacgtatttattaacttctcctagtattagtaattatcatggctgtcatggcgcattaacggaataaagggtgtgcttaaatcgggccattttgcgtaataagaaaaaggattaattatgagcgaattgaattaataataaggtaatagatttacattagaaaatgaaaggggattttatgcgtgagaatgttacagtctatcccggcattgccagtcggggatattaaaaagagtataggtttttattgcgataaactaggtttcactttggttcaccatgaagatggattcgcagttctaatgtgtaatgaggttcggattcatctatgggaggcaagtgatgaaggctggcgctctcgtagtaatgattcaccggtttgtacaggtgcggagtcgtttattgctggtactgctagttgccgcattgaagtagagggaattgatgaattatatcaacatattaagcctttgggcattttgcaccccaatacatcattaaaagatcagtggtgggatgaacgagactttgcagtaattgatcccgacaacaatttgattagcttttttcaacaaataaaaagctaaaatctattattaatctgttcagcaatcgggcgcgattgctgaataaaagatacgagagacctctcttgtatcttttttattttgagtggttttgtccgttacactagaaaaccgaaagacaataaaaattttattcttgctgagtctggctttcggtaagctagacaaaacggacaaaataaaaattggcaagggtttaaaggtggagattttttgagtgatcttctcaaaaaatactacctgtcccttgctgatttttaaacgagcacgagagcaaaacccccctttgctgaggtggcagagggcaggtttttttgtttcttttttctcgtaaaaaaaagaaaggtcttaaaggttttatggttttggtcggcactgccgacagcctcgcagagcacacactttatgaatataaagtatagtgtgttatactttacttggaagtggttgccggaaagagcgaaaatgcctcacatttgtgccacctaaaaaggagcgattta

SEQ ID NO: 4 - DNA, 8716 bp, Artificial Sequence
  pMA5-HpaII-UGT
catgatcttctcaaaaaatactacctgtcccttgctgatttttaaacgagcacgagagcaaaacccccctttgctgaggtggcagagggcaggtttttttgtttcttttttctcgtaaaaaaaagaaaggtcttaaaggttttatggttttggtcggcactgccgacagcctcgcagagcacacactttatgaatataaagtatagtgtgttatactttacttggaagtggttgccggaaagagcgaaaatgcctcacatttgtgccacctaaaaaggagcgatttacatatgggcaccgaggtgaccgttcacaaaaacaccctgcgtgtgctgatgttcccgtggctggcgtacggtcacatcagcccgtttctgaacgttgcgaagaaactggtggatcgtggcttcctgatctacctgtgcagcaccgcgattaacctgaaaagcaccatcaagaaaattccggagaagtatagcgacagcatccagctgattgaactgcacctgccggagctgccggaactgccgccgcactaccacaccaccaacggtctgccgccgcacctgaaccacaccctgcaaaaggcgctgaaaatgagcaagccgaacttcagcaaaatcctgcaaaacctgaagccggacctggttatttatgacctgctgcaacaatgggcggagggcgttgcgaacgaacaaaacattccggcggtgaagctgctgaccagcggtgcggcggttctgagctacttctttaacctggttaagaaaccgggcgtggagttcccgtttccggcgatttatctgcgtaaaaacgagctggaaaagatgagcgagctgctggcgcagagcgcgaaggataaagaaccggatggtgtggatccgtttgcggatggtaacatgcaagttatgctgatgagcaccagccgtatcatcgaagcgaagtacatcgattactttagcggtctgagcaactggaaggtggttccggtgggtccgccggttcaggacccgatcgcggacgatgcggacgagatggaactgattgattggctgggtaagaaagacgagaacagcaccgtgttcgttagctttggcagcgagtacttcctgagcaaagaagatcgtgaggaaatcgcgtttggtctggaactgagcaacgttaacttcatttgggtggcgcgttttccgaaaggcgaggaacagaacctggaggacgcgctgccgaagggcttcctggaacgtatcggtgatcgtggccgtgtgctggacaagtttgcgccgcaaccgcgtatcctgaaccacccgagcaccggtggcttcattagccactgcggttggaacagcgtgatggaaagcgttgattttggcgtgccgatcattgcgatgccgatccacctggaccagccgatgaacgcgcgtctgattgtggagctgggtgtggcggttgaaatcgttcgtgacgattatggcaaaattcaccgtgaggaaatcgcggagattctgaaagatgttatcgcgggcaagagcggcgaaaacctgaaggcgaaaatgcgtgacatcagcaaaaacctgaagagcattcgtgacgaggaaatggataccgcggcggaggaactgattcaactgtgcaagaacagcccgaagctgaaataaggatcctctagagtcgagctcaagctagcttggtacgtaccagatctgagatcacgcgttctagaggtcgaaattcacctcgaaagcaagctgataaaccgatacaattaaaggctccttttggagcctttttttttggagattttcaacgtgaaaaaattattattcgcaattccaagctaattcacctcgaaagcaagctgataaaccgatacaattaaaggctccttttggagcctttttttttggagattttcaacgtgaaaaaattattattcgcaattccaagctctgcctcgcgcgtttcggtgatgacggtgaaaacctctgacacatgcagctcccggagacggtcacagcttgtctgtaagcggatgcagatcacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgactgcaatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaagtactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggcatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacgaaattccggatgagcattcatcaggcgggcaagaatgtgaataaaggccggataaaacttgtgcttatttttctttacggtctttaaaaaggccgtaatatccagctaaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcgaagacgaaagggcatcgcgcgcggggaattcccgggagagctcgatatcgcatgcggtacctctagaagaagcttggagacaaggtaaaggataaaacagcacaattccaagaaaaacacgatttagaacctaaaaagaacgaatttgaactaactcataaccgagaggtaaaaaaagaacgaagtcgagatcagggaatgagtttataaaataaaaaaagcacctgaaaaggtgtctttttttgatggttttgaacttgttctttcttatcttgatacatatagaaataacgtcatttttattttagttgctgaaaggtgcgttgaagtgttggtatgtatgtgttttaaagtattgaaaacccttaaaattggttgcacagaaaaaccccatctgttaaagttataagtgactaaacaaataactaaatagatgggggtttcttttaatattatgtgtcctaatagtagcatttattcagatgaaaaatcaagggttttagtggacaagacaaaaagtggaaaagtgagaccatggagagaaaagaaaatcgctaatgttgattactttgaacttctgcatattcttgaatttaaaaaggctgaaagagtaaaagattgtgctgaaatattagagtataaacaaaatcgtgaaacaggcgaaagaaagttgtatcgagtgtggttttgtaaatccaggctttgtccaatgtgcaactggaggagagcaatgaaacatggcattcagtcacaaaaggttgttgctgaagttattaaacaaaagccaacagttcgttggttgtttctcacattaacagttaaaaatgtttatgatggcgaagaattaaataagagtttgtcagatatggctcaaggatttcgccgaatgatgcaatataaaaaaattaataaaaatcttgttggttttatgcgtgcaacggaagtgacaataaataataaagataattcttataatcagcacatgcatgtattggtatgtgtggaaccaacttattttaagaatacagaaaactacgtgaatcaaaaacaatggattcaattttggaaaaaggcaatgaaattagactatgatccaaatgtaaaagttcaaatgattcgaccgaaaaataaatataaatcggatatacaatcggcaattgacgaaactgcaaaatatcctgtaaaggatacggattttatgaccgatgatgaagaaaagaatttgaaacgtttgtctgatttggaggaaggtttacaccgtaaaaggttaatctcctatggtggtttgttaaaagaaatacataaaaaattaaaccttgatgacacagaagaaggcgatttgattcatacagatgatgacgaaaaagccgatgaagatggattttctattattgcaatgtggaattgggaacggaaaaattattttattaaagagtagttcaacaaacgggccagtttgttgaagattagatgctataattgttattaaaaggattgaaggatgcttaggaagacgagttattaatagctgaataagaacggtgctctccaaatattcttatttagaaaagcaaatctaaaattatctgaaaagggaatgagaatagtgaatggaccaataataatgactagagaagaaagaatgaagattgttcatgaaattaaggaacgaatattggataaatatggggatgatgttaaggctattggtgtttatggctctcttggtcgtcagactgatgggccctattcggatattgagatgatgtgtgtcatgtcaacagaggaagcagagttcagccatgaatggacaaccggtgagtggaaggtggaagtgaattttgatagcgaagagattctactagattatgcatctcaggtggaatcagattggccgcttacacatggtcaatttttctctattttgccgatttatgattcaggtggatacttagagaaagtgtatcaaactgctaaatcggtagaagcccaaacgttccacgatgcgatttgtgcccttatcgtagaagagctgtttgaatatgcaggcaaatggcgtaatattcgtgtgcaaggaccgacaacatttctaccatccttgactgtacaggtagcaatggcaggtgccatgttgattggtctgcatcatcgcatctgttatacgacgagcgcttcggtcttaactgaagcagttaagcaatcagatcttccttcaggttatgaccatctgtgccagttcgtaatgtctggtcaactttccgactctgagaaacttctggaatcgctagagaatttctggaatgggattcaggagtggacagaacgacacggatatatagtggatgtgtcaaaacgcataccattttgaacgatgacctctaataattgttaatcatgttggttacgtatttattaacttctcctagtattagtaattatcatggctgtcatggcgcattaacggaataaagggtgtgcttaaatcgggccattttgcgtaataagaaaaaggattaattatgagcgaattgaattaataataaggtaatagatttacattagaaaatgaaaggggattttatgcgtgagaatgttacagtctatcccggcattgccagtcggggatattaaaaagagtataggtttttattgcgataaactaggtttcactttggttcaccatgaagatggattcgcagttctaatgtgtaatgaggttcggattcatctatgggaggcaagtgatgaaggctggcgctctcgtagtaatgattcaccggtttgtacaggtgcggagtcgtttattgctggtactgctagttgccgcattgaagtagagggaattgatgaattatatcaacatattaagcctttgggcattttgcaccccaatacatcattaaaagatcagtggtgggatgaacgagactttgcagtaattgatcccgacaacaatttgattagcttttttcaacaaataaaaagctaaaatctattattaatctgttcagcaatcgggcgcgattgctgaataaaagatacgagagacctctcttgtatcttttttattttgagtggttttgtccgttacactagaaaaccgaaagacaataaaaattttattcttgctgagtctggctttcggtaagctagacaaaacggacaaaataaaaattggcaagggtttaaaggtggagattttttgagtgatcttctcaaaaaatactacctgtcccttgctgatttttaaacgagcacgagagcaaaacccccctttgctgaggtggcagagggcaggtttttttgtttcttttttctcgtaaaaaaaagaaaggtcttaaaggttttatggttttggtcggcactgccgacagcctcgcagagcacacactttatgaatataaagtatagtgtgttatactttacttggaagtggttgccggaaagagcgaaaatgcctcacatttgtgccacctaaaaaggagcgattta

SEQ ID NO: 5 - DNA, 8732 bp, Artificial Sequence
  pMA5-P43-UGT
cattgataggtggtatgttttcgcttgaacttttaaatacagccattgaacatacggttgatttaataactgacaaacatcaccctcttgctaaagcggccaaggacgctgccgccggggctgtttgcgtttttgccgtgatttcgtgtatcattggtttacttatttttttgccaaagctgtaatggctgaaaattcttacatttattttacatttttagaaatgggcgtgaaaaaaagcgcgcgattatgtaaaatataaagtgatagcggtaccattataggtaagagaggaatgtacaccatatgggcaccgaggtgaccgttcacaaaaacaccctgcgtgtgctgatgttcccgtggctggcgtacggtcacatcagcccgtttctgaacgttgcgaagaaactggtggatcgtggcttcctgatctacctgtgcagcaccgcgattaacctgaaaagcaccatcaagaaaattccggagaagtatagcgacagcatccagctgattgaactgcacctgccggagctgccggaactgccgccgcactaccacaccaccaacggtctgccgccgcacctgaaccacaccctgcaaaaggcgctgaaaatgagcaagccgaacttcagcaaaatcctgcaaaacctgaagccggacctggttatttatgacctgctgcaacaatgggcggagggcgttgcgaacgaacaaaacattccggcggtgaagctgctgaccagcggtgcggcggttctgagctacttctttaacctggttaagaaaccgggcgtggagttcccgtttccggcgatttatctgcgtaaaaacgagctggaaaagatgagcgagctgctggcgcagagcgcgaaggataaagaaccggatggtgtggatccgtttgcggatggtaacatgcaagttatgctgatgagcaccagccgtatcatcgaagcgaagtacatcgattactttagcggtctgagcaactggaaggtggttccggtgggtccgccggttcaggacccgatcgcggacgatgcggacgagatggaactgattgattggctgggtaagaaagacgagaacagcaccgtgttcgttagctttggcagcgagtacttcctgagcaaagaagatcgtgaggaaatcgcgtttggtctggaactgagcaacgttaacttcatttgggtggcgcgttttccgaaaggcgaggaacagaacctggaggacgcgctgccgaagggcttcctggaacgtatcggtgatcgtggccgtgtgctggacaagtttgcgccgcaaccgcgtatcctgaaccacccgagcaccggtggcttcattagccactgcggttggaacagcgtgatggaaagcgttgattttggcgtgccgatcattgcgatgccgatccacctggaccagccgatgaacgcgcgtctgattgtggagctgggtgtggcggttgaaatcgttcgtgacgattatggcaaaattcaccgtgaggaaatcgcggagattctgaaagatgttatcgcgggcaagagcggcgaaaacctgaaggcgaaaatgcgtgacatcagcaaaaacctgaagagcattcgtgacgaggaaatggataccgcggcggaggaactgattcaactgtgcaagaacagcccgaagctgaaataaggatcctctagagtcgagctcaagctagcttggtacgtaccagatctgagatcacgcgttctagaggtcgaaattcacctcgaaagcaagctgataaaccgatacaattaaaggctccttttggagcctttttttttggagattttcaacgtgaaaaaattattattcgcaattccaagctaattcacctcgaaagcaagctgataaaccgatacaattaaaggctccttttggagcctttttttttggagattttcaacgtgaaaaaattattattcgcaattccaagctctgcctcgcgcgtttcggtgatgacggtgaaaacctctgacacatgcagctcccggagacggtcacagcttgtctgtaagcggatgcagatcacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgactgcaatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaagtactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggcatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacgaaattccggatgagcattcatcaggcgggcaagaatgtgaataaaggccggataaaacttgtgcttatttttctttacggtctttaaaaaggccgtaatatccagctaaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcgaagacgaaagggcatcgcgcgcggggaattcccgggagagctcgatatcgcatgcggtacctctagaagaagcttggagacaaggtaaaggataaaacagcacaattccaagaaaaacacgatttagaacctaaaaagaacgaatttgaactaactcataaccgagaggtaaaaaaagaacgaagtcgagatcagggaatgagtttataaaataaaaaaagcacctgaaaaggtgtctttttttgatggttttgaacttgttctttcttatcttgatacatatagaaataacgtcatttttattttagttgctgaaaggtgcgttgaagtgttggtatgtatgtgttttaaagtattgaaaacccttaaaattggttgcacagaaaaaccccatctgttaaagttataagtgactaaacaaataactaaatagatgggggtttcttttaatattatgtgtcctaatagtagcatttattcagatgaaaaatcaagggttttagtggacaagacaaaaagtggaaaagtgagaccatggagagaaaagaaaatcgctaatgttgattactttgaacttctgcatattcttgaatttaaaaaggctgaaagagtaaaagattgtgctgaaatattagagtataaacaaaatcgtgaaacaggcgaaagaaagttgtatcgagtgtggttttgtaaatccaggctttgtccaatgtgcaactggaggagagcaatgaaacatggcattcagtcacaaaaggttgttgctgaagttattaaacaaaagccaacagttcgttggttgtttctcacattaacagttaaaaatgtttatgatggcgaagaattaaataagagtttgtcagatatggctcaaggatttcgccgaatgatgcaatataaaaaaattaataaaaatcttgttggttttatgcgtgcaacggaagtgacaataaataataaagataattcttataatcagcacatgcatgtattggtatgtgtggaaccaacttattttaagaatacagaaaactacgtgaatcaaaaacaatggattcaattttggaaaaaggcaatgaaattagactatgatccaaatgtaaaagttcaaatgattcgaccgaaaaataaatataaatcggatatacaatcggcaattgacgaaactgcaaaatatcctgtaaaggatacggattttatgaccgatgatgaagaaaagaatttgaaacgtttgtctgatttggaggaaggtttacaccgtaaaaggttaatctcctatggtggtttgttaaaagaaatacataaaaaattaaaccttgatgacacagaagaaggcgatttgattcatacagatgatgacgaaaaagccgatgaagatggattttctattattgcaatgtggaattgggaacggaaaaattattttattaaagagtagttcaacaaacgggccagtttgttgaagattagatgctataattgttattaaaaggattgaaggatgcttaggaagacgagttattaatagctgaataagaacggtgctctccaaatattcttatttagaaaagcaaatctaaaattatctgaaaagggaatgagaatagtgaatggaccaataataatgactagagaagaaagaatgaagattgttcatgaaattaaggaacgaatattggataaatatggggatgatgttaaggctattggtgtttatggctctcttggtcgtcagactgatgggccctattcggatattgagatgatgtgtgtcatgtcaacagaggaagcagagttcagccatgaatggacaaccggtgagtggaaggtggaagtgaattttgatagcgaagagattctactagattatgcatctcaggtggaatcagattggccgcttacacatggtcaatttttctctattttgccgatttatgattcaggtggatacttagagaaagtgtatcaaactgctaaatcggtagaagcccaaacgttccacgatgcgatttgtgcccttatcgtagaagagctgtttgaatatgcaggcaaatggcgtaatattcgtgtgcaaggaccgacaacatttctaccatccttgactgtacaggtagcaatggcaggtgccatgttgattggtctgcatcatcgcatctgttatacgacgagcgcttcggtcttaactgaagcagttaagcaatcagatcttccttcaggttatgaccatctgtgccagttcgtaatgtctggtcaactttccgactctgagaaacttctggaatcgctagagaatttctggaatgggattcaggagtggacagaacgacacggatatatagtggatgtgtcaaaacgcataccattttgaacgatgacctctaataattgttaatcatgttggttacgtatttattaacttctcctagtattagtaattatcatggctgtcatggcgcattaacggaataaagggtgtgcttaaatcgggccattttgcgtaataagaaaaaggattaattatgagcgaattgaattaataataaggtaatagatttacattagaaaatgaaaggggattttatgcgtgagaatgttacagtctatcccggcattgccagtcggggatattaaaaagagtataggtttttattgcgataaactaggtttcactttggttcaccatgaagatggattcgcagttctaatgtgtaatgaggttcggattcatctatgggaggcaagtgatgaaggctggcgctctcgtagtaatgattcaccggtttgtacaggtgcggagtcgtttattgctggtactgctagttgccgcattgaagtagagggaattgatgaattatatcaacatattaagcctttgggcattttgcaccccaatacatcattaaaagatcagtggtgggatgaacgagactttgcagtaattgatcccgacaacaatttgattagcttttttcaacaaataaaaagctaaaatctattattaatctgttcagcaatcgggcgcgattgctgaataaaagatacgagagacctctcttgtatcttttttattttgagtggttttgtccgttacactagaaaaccgaaagacaataaaaattttattcttgctgagtctggctttcggtaagctagacaaaacggacaaaataaaaattggcaagggtttaaaggtggagattttttgagtgatcttctcaaaaaatactacctgtcccttgctgatttttaaacgagcacgagagcaaaacccccctttgctgaggtggcagagggcaggtttttttgtttcttttttctcgtaaaaaaaagaaaggtcttaaaggttttatggttttggtcggcactgccgacagcctcgcagagcacacactttatgaatataaagtatagtgtgttatactttacttggaagtggttgccggaaagagcgaaaatgcctcacatttgtgccacctaaaaaggagcgattta

SEQ ID NO: 6 - DNA, 8707 bp, Artificial Sequence
  pMA5-P43t-UGT
cattgataggtggtatgttttcgcttgaacttttaaatacagccattgaacatacggttgatttaataactgacaaacatcaccctcttgctaaagcggccaaggacgctgccgccggggctgtttgcgtttttgccgtgatttcgtgtatcattggtttacttatttttttgccaaagctgtaatggctgaaaattcttacatttattttacatttttagaaatgggcgtgaaaaaaagcgcgcgattatgtaaaatataaagtgatagcggtaccacatatgggcaccgaggtgaccgttcacaaaaacaccctgcgtgtgctgatgttcccgtggctggcgtacggtcacatcagcccgtttctgaacgttgcgaagaaactggtggatcgtggcttcctgatctacctgtgcagcaccgcgattaacctgaaaagcaccatcaagaaaattccggagaagtatagcgacagcatccagctgattgaactgcacctgccggagctgccggaactgccgccgcactaccacaccaccaacggtctgccgccgcacctgaaccacaccctgcaaaaggcgctgaaaatgagcaagccgaacttcagcaaaatcctgcaaaacctgaagccggacctggttatttatgacctgctgcaacaatgggcggagggcgttgcgaacgaacaaaacattccggcggtgaagctgctgaccagcggtgcggcggttctgagctacttctttaacctggttaagaaaccgggcgtggagttcccgtttccggcgatttatctgcgtaaaaacgagctggaaaagatgagcgagctgctggcgcagagcgcgaaggataaagaaccggatggtgtggatccgtttgcggatggtaacatgcaagttatgctgatgagcaccagccgtatcatcgaagcgaagtacatcgattactttagcggtctgagcaactggaaggtggttccggtgggtccgccggttcaggacccgatcgcggacgatgcggacgagatggaactgattgattggctgggtaagaaagacgagaacagcaccgtgttcgttagctttggcagcgagtacttcctgagcaaagaagatcgtgaggaaatcgcgtttggtctggaactgagcaacgttaacttcatttgggtggcgcgttttccgaaaggcgaggaacagaacctggaggacgcgctgccgaagggcttcctggaacgtatcggtgatcgtggccgtgtgctggacaagtttgcgccgcaaccgcgtatcctgaaccacccgagcaccggtggcttcattagccactgcggttggaacagcgtgatggaaagcgttgattttggcgtgccgatcattgcgatgccgatccacctggaccagccgatgaacgcgcgtctgattgtggagctgggtgtggcggttgaaatcgttcgtgacgattatggcaaaattcaccgtgaggaaatcgcggagattctgaaagatgttatcgcgggcaagagcggcgaaaacctgaaggcgaaaatgcgtgacatcagcaaaaacctgaagagcattcgtgacgaggaaatggataccgcggcggaggaactgattcaactgtgcaagaacagcccgaagctgaaataaggatcctctagagtcgagctcaagctagcttggtacgtaccagatctgagatcacgcgttctagaggtcgaaattcacctcgaaagcaagctgataaaccgatacaattaaaggctccttttggagcctttttttttggagattttcaacgtgaaaaaattattattcgcaattccaagctaattcacctcgaaagcaagctgataaaccgatacaattaaaggctccttttggagcctttttttttggagattttcaacgtgaaaaaattattattcgcaattccaagctctgcctcgcgcgtttcggtgatgacggtgaaaacctctgacacatgcagctcccggagacggtcacagcttgtctgtaagcggatgcagatcacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgactgcaatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaagtactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggcatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacgaaattccggatgagcattcatcaggcgggcaagaatgtgaataaaggccggataaaacttgtgcttatttttctttacggtctttaaaaaggccgtaatatccagctaaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcgaagacgaaagggcatcgcgcgcggggaattcccgggagagctcgatatcgcatgcggtacctctagaagaagcttggagacaaggtaaaggataaaacagcacaattccaagaaaaacacgatttagaacctaaaaagaacgaatttgaactaactcataaccgagaggtaaaaaaagaacgaagtcgagatcagggaatgagtttataaaataaaaaaagcacctgaaaaggtgtctttttttgatggttttgaacttgttctttcttatcttgatacatatagaaataacgtcatttttattttagttgctgaaaggtgcgttgaagtgttggtatgtatgtgttttaaagtattgaaaacccttaaaattggttgcacagaaaaaccccatctgttaaagttataagtgactaaacaaataactaaatagatgggggtttcttttaatattatgtgtcctaatagtagcatttattcagatgaaaaatcaagggttttagtggacaagacaaaaagtggaaaagtgagaccatggagagaaaagaaaatcgctaatgttgattactttgaacttctgcatattcttgaatttaaaaaggctgaaagagtaaaagattgtgctgaaatattagagtataaacaaaatcgtgaaacaggcgaaagaaagttgtatcgagtgtggttttgtaaatccaggctttgtccaatgtgcaactggaggagagcaatgaaacatggcattcagtcacaaaaggttgttgctgaagttattaaacaaaagccaacagttcgttggttgtttctcacattaacagttaaaaatgtttatgatggcgaagaattaaataagagtttgtcagatatggctcaaggatttcgccgaatgatgcaatataaaaaaattaataaaaatcttgttggttttatgcgtgcaacggaagtgacaataaataataaagataattcttataatcagcacatgcatgtattggtatgtgtggaaccaacttattttaagaatacagaaaactacgtgaatcaaaaacaatggattcaattttggaaaaaggcaatgaaattagactatgatccaaatgtaaaagttcaaatgattcgaccgaaaaataaatataaatcggatatacaatcggcaattgacgaaactgcaaaatatcctgtaaaggatacggattttatgaccgatgatgaagaaaagaatttgaaacgtttgtctgatttggaggaaggtttacaccgtaaaaggttaatctcctatggtggtttgttaaaagaaatacataaaaaattaaaccttgatgacacagaagaaggcgatttgattcatacagatgatgacgaaaaagccgatgaagatggattttctattattgcaatgtggaattgggaacggaaaaattattttattaaagagtagttcaacaaacgggccagtttgttgaagattagatgctataattgttattaaaaggattgaaggatgcttaggaagacgagttattaatagctgaataagaacggtgctctccaaatattcttatttagaaaagcaaatctaaaattatctgaaaagggaatgagaatagtgaatggaccaataataatgactagagaagaaagaatgaagattgttcatgaaattaaggaacgaatattggataaatatggggatgatgttaaggctattggtgtttatggctctcttggtcgtcagactgatgggccctattcggatattgagatgatgtgtgtcatgtcaacagaggaagcagagttcagccatgaatggacaaccggtgagtggaaggtggaagtgaattttgatagcgaagagattctactagattatgcatctcaggtggaatcagattggccgcttacacatggtcaatttttctctattttgccgatttatgattcaggtggatacttagagaaagtgtatcaaactgctaaatcggtagaagcccaaacgttccacgatgcgatttgtgcccttatcgtagaagagctgtttgaatatgcaggcaaatggcgtaatattcgtgtgcaaggaccgacaacatttctaccatccttgactgtacaggtagcaatggcaggtgccatgttgattggtctgcatcatcgcatctgttatacgacgagcgcttcggtcttaactgaagcagttaagcaatcagatcttccttcaggttatgaccatctgtgccagttcgtaatgtctggtcaactttccgactctgagaaacttctggaatcgctagagaatttctggaatgggattcaggagtggacagaacgacacggatatatagtggatgtgtcaaaacgcataccattttgaacgatgacctctaataattgttaatcatgttggttacgtatttattaacttctcctagtattagtaattatcatggctgtcatggcgcattaacggaataaagggtgtgcttaaatcgggccattttgcgtaataagaaaaaggattaattatgagcgaattgaattaataataaggtaatagatttacattagaaaatgaaaggggattttatgcgtgagaatgttacagtctatcccggcattgccagtcggggatattaaaaagagtataggtttttattgcgataaactaggtttcactttggttcaccatgaagatggattcgcagttctaatgtgtaatgaggttcggattcatctatgggaggcaagtgatgaaggctggcgctctcgtagtaatgattcaccggtttgtacaggtgcggagtcgtttattgctggtactgctagttgccgcattgaagtagagggaattgatgaattatatcaacatattaagcctttgggcattttgcaccccaatacatcattaaaagatcagtggtgggatgaacgagactttgcagtaattgatcccgacaacaatttgattagcttttttcaacaaataaaaagctaaaatctattattaatctgttcagcaatcgggcgcgattgctgaataaaagatacgagagacctctcttgtatcttttttattttgagtggttttgtccgttacactagaaaaccgaaagacaataaaaattttattcttgctgagtctggctttcggtaagctagacaaaacggacaaaataaaaattggcaagggtttaaaggtggagattttttgagtgatcttctcaaaaaatactacctgtcccttgctgatttttaaacgagcacgagagcaaaacccccctttgctgaggtggcagagggcaggtttttttgtttcttttttctcgtaaaaaaaagaaaggtcttaaaggttttatggttttggtcggcactgccgacagcctcgcagagcacacactttatgaatataaagtatagtgtgttatactttacttggaagtggttgccggaaagagcgaaaatgcctcacatttgtgccacctaaaaaggagcgattta

SEQ ID NO: 7 - DNA, 38 bp, Artificial Sequence
  Primer
ggagcgatttacatatggcaacaaatttaagagtcctg

SEQ ID NO: 8 - DNA, 41 bp, Artificial Sequence
  Primer
gctcgactctagaggatccttattttgatttgttagaattg

SEQ ID NO: 9 - DNA, 35 bp, Artificial Sequence
  Primer
ggagcgatttacatatggcaacaaatttaagagtc

SEQ ID NO: 10 - DNA, 43 bp, Artificial Sequence
  Primer
ctaaaaaggagcgatttacatgatcttctcaaaaaatactacc

SEQ ID NO: 11 - DNA, 32 bp, Artificial Sequence
  Primer
gttgccatatgtaaatcgctcctttttaggtg

SEQ ID NO: 12 - DNA, 35 bp, Artificial Sequence
  Primer
ggaatgtacaccatatggcaacaaatttaagagtc

SEQ ID NO: 13 - DNA, 40 bp, Artificial Sequence
  Primer
ctaaaaaggagcgatttacattgataggtggtatgttttc

SEQ ID NO: 14 - DNA, 31 bp, Artificial Sequence
  Primer
gttgccatatggtgtacattcctctcttacc

SEQ ID NO: 15 - DNA, 37 bp, Artificial Sequence
  Primer
gatagcggtaccacatatggcaacaaatttaagagtc

SEQ ID NO: 16 - DNA, 32 bp, Artificial Sequence
  Primer
gttgccatatgtggtaccgctatcactttata

SEQ ID NO: 17 - DNA, 40 bp, Artificial Sequence
  Primer
caaataaggagtgtcaagaggcaagggtttaaaggtggag

SEQ ID NO: 18 - DNA, 40 bp, Artificial Sequence
  Primer
gtcccgtctagccttgcccgagctcgactctagaggatcc

SEQ ID NO: 19 - DNA, 19 bp, Artificial Sequence
  Primer
gggcaaggctagacgggac

SEQ ID NO: 20 - DNA, 19 bp, Artificial Sequence
  Primer
tcttgacactccttatttg